(19) United States
(12) Patent Application Publication
(10) Pub. No.: US 2013/0226214 A1
(43) Pub. Date: Aug. 29, 2013

(54) HAIR IMPLANTER WITH IMPROVED STRUCTURE OF NEEDLE

(71) Applicant: DENSO CORPORATION, (US)

(72) Inventor: Hideki OKUDA, Nagoya (JP)

(73) Assignee: DENSO CORPORATION, Kariya-city (JP)

(21) Appl. No.: 13/775,440

(22) Filed: Feb. 25, 2013

(30) Foreign Application Priority Data
Feb. 29, 2012 (JP) 2012-043430

(51) Int. Cl. A61B 17/34

(52) U.S. Cl. CPC A61B 17/3468
USPC 606/187

(57) ABSTRACT

A hair implanter is equipped with a tube in which a shaft is disposed movable in an axial direction thereof, a tapered hollow needle extending from a tip end of the tube, and slits extending from a tip of the needle in an axial direction thereof to divide a body of the needle into a plurality of needle strips. The needle strips are so shaped that when the shaft is pushed, it contacts inner surfaces of the needle strips, and when the shaft is further advanced in sliding contact with the inner surfaces of the needle strips, the needle strips will elastically bend radially outwardly. Each of the slits extends toward the base end of the tube across a point at which the shaft contacts the inner surfaces of the needle strips. This achieves smooth opening of the tip of the needle with a small degree of force pushing the shaft.

### CROSS REFERENCE TO RELATED DOCUMENT

The present application claims the benefit of priority of Japanese Patent Application No. 2012-43430 filed on Feb. 29, 2012, the disclosure of which is incorporated herein by reference.

### BACKGROUND OF THE INVENTION

1. Technical Field

This disclosure relates generally to a hair implanter for use in implanting a hair graft into a recipient site.

2. Background Art

Japanese Patent No. 3035668 (corresponding to U.S. Pat. No. 5,782,843) teaches hair root transplantation surgery techniques of removing a scalp graft containing hair roots using a hollow needle and transplanting it to, for example, an incision in the skin of the scalp.

Recently, a hair implantation mechanism has been proposed which is equipped with an assembly of a plurality of flexible needle strips disposed on an end thereof. The needle strips can be opened for holding a skin graft. The hair implantation mechanism, however, needs to achieve smooth opening or closing of the needle strips. If not, it may result in breakage of the needle strips or physical damage to or splitting of the skin graft.

### SUMMARY

It is therefore an object to provide an improved structure of a hair implanter equipped with needle strips which are geometrically designed to be opened or closed smoothly.

According to one aspect of an embodiment, there is provided a hair implanter which may be employed in harvesting a hair graft from a physiological tissue or implanting the hair graft into a recipient site. The hair implanter comprises: (a) a hollow cylindrical tube in which a shaft is to be moved in an axial direction of the tube, the tube having a top end and a base end opposite the top end; (b) a hollow cylindrical needle having a tip and a base end opposite the tip, the needle extending at the base end thereof from the tip end of the tube, the needle tapering toward the tip; and (c) a plurality of slits extending from the tip of the needle in an axial direction of the needle to divide a body of the needle into a plurality of needle strips. The needle strips are so shaped that when the shaft is moved toward the top of the tube, contacts inner surfaces of the needle strips, and is further advanced in sliding contact with the inner surfaces of the needle strips, the needle strips will elastically bend radially outwardly. Each of the slits extends toward the base end of the tube across a point at which the shaft contacts the inner surfaces of the needle strips. This causes the needle strips to bend or expand radially outwardly to open the tip of the needle in a leverage mode where the point, of contact between the shaft contacts the inner surface of each of the needle strops is an effort point, thereby opening the tip of the needle smoothly by thrusting the shaft forward with a small degree of force.

In a preferred mode of the embodiment, each of the slits may have a given length with a top end and a base end and also have a width which gradually narrows from the base end to the top end of the slits. This facilitates the ease with which the needle is inserted into, for example, skin of a donor.

The slits may be so shaped that portions of the needle strips which are closer to the top end thereof than to the base end thereof are placed in contact with each other.

The portions of the needle strips placed in touch with each other may be middle portions of the needle strips excluding the top end of the needle.

The portions of the needle strips each may have a given length extending in the axial direction of the needle.

### BRIEF DESCRIPTION OF THE DRAWINGS

The present invention will be understood more fully from the detailed description given hereinbelow and from the accompanying drawings of the preferred embodiments of the invention, which, however, should not be taken to limit the invention to the specific embodiments but are for the purpose of explanation and understanding only.

In the drawings:

FIGS. 1(a), 1(b), and 1(c) are longitudinal sectional views which illustrate a hair implanter according to a first embodiment;

FIG. 2 is a flowchart of a sequence of steps of producing the hair implanter of FIGS. 1(a) to 1(c);

FIGS. 3(a), 3(b), 3(c), and 3(d) are longitudinal sectional views which illustrates a sequence of production steps of the hair implanter of FIGS. 1(a) to 1(c);

FIG. 4 is a longitudinal sectional view which illustrates a hair transplanter in which the hair implanter, as shown in FIGS. 1(a) to 1(c), is installed;

FIGS. 5(a), 5(b), 5(c), 5(d), 5(e), 5(f), and 5(g) are longitudinal sectional views which demonstrate how to harvest a hair graft using the hair transplanter of FIG. 4 and implant the hair graft in a recipient site;

FIGS. 6(a), 6(b), and 6(c) are longitudinal sectional views which illustrate a hair implanter according to a second embodiment;

FIGS. 7(a), 7(b), 7(c), and 7(d) are longitudinal sectional views which illustrate a sequence of production steps of the hair implanter of FIGS. 6(a) to 6(c); and

FIGS. 8(a), 8(b), and 8(c) are longitudinal sectional views which illustrate a hair implanter according to a third embodiment.

### DESCRIPTION OF THE PREFERRED EMBODIMENTS

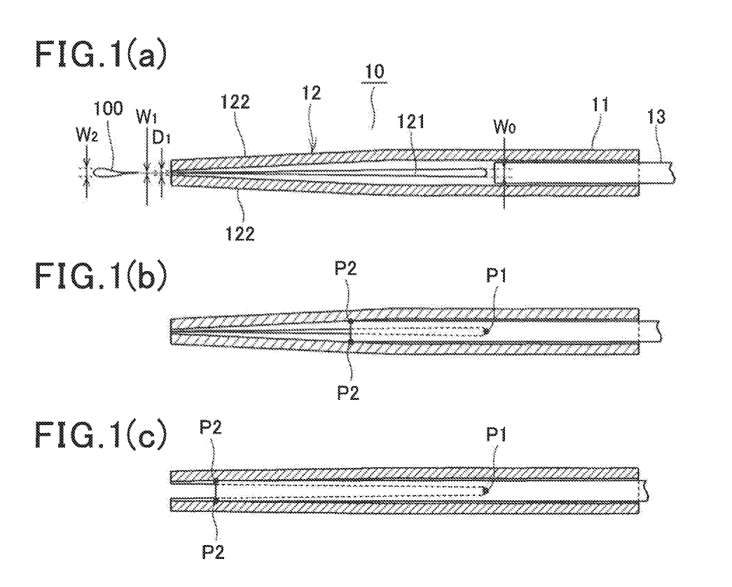

Referring to the drawings, wherein like reference numbers refer to like parts in several views, particularly to FIGS. 1(a) to 5(g), there is shown a hair implanter 10 according to the first embodiment. The hair implanter 10 is made of metallic material such as stainless steel and includes a straight hollow cylinder or tube 11, and a tapered hollow cylindrical needle 12 formed integrally with the hollow tube 11.

The hollow tube 11 is cylindrical and formed to have an inner diameter which is slightly greater than an outer diameter of a shaft 13 to retain the shaft 13 to be slidable in the tube 11 in an axial direction (i.e., a lengthwise direction) thereof. In other words, the tube 11 has formed therein a cylindrical chamber in which the shaft 13 is held and works as a guide to guide the back-and-forth motion of the shaft 13 in the longitudinal direction thereof.

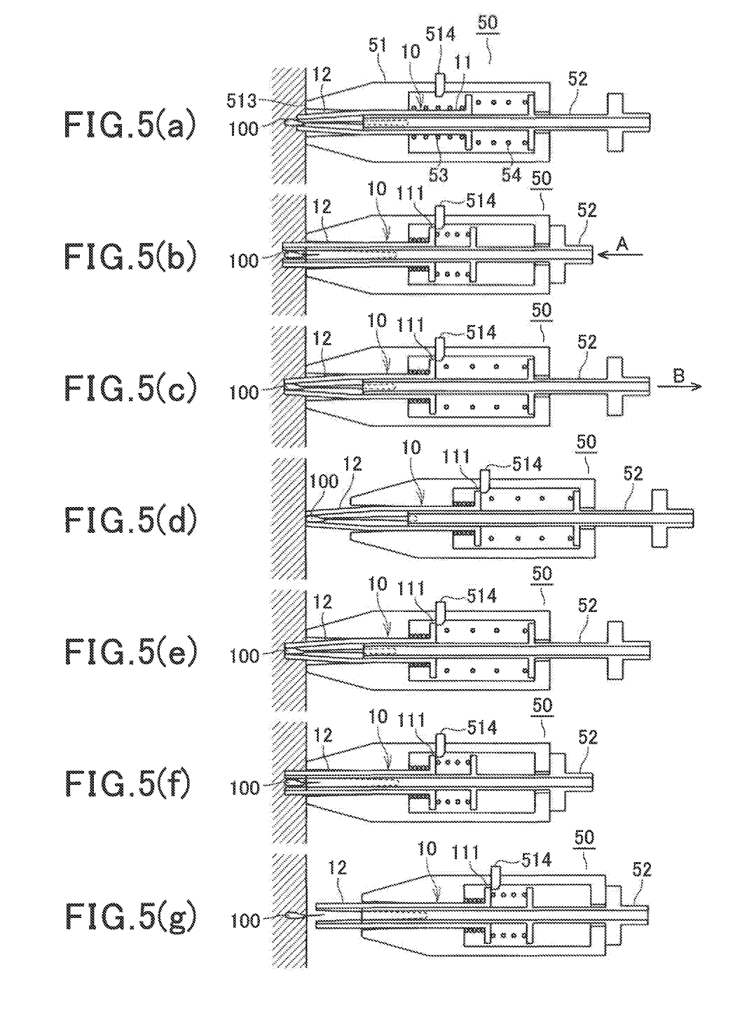

The needle 12 has a given length with a top end and a base end opposite the top end. The top end will also be referred to as a tip below. The needle 12 extends form the top end of the tube 11 in alignment with the tube 11, but may be oriented out of alignment with the tube 11. A combination of the needle 12 and the tube 11 defines a hollow cylindrical body of the hair implanter 10. The needle 12 is spine-like and tapers from the top end of the tube 11 toward the tip thereof. In other words, the outer diameter of the needle 12 decreases gradually to the top thereof. The needle 12 has slits 121 extending straight from the top end of the needle 12 toward the base end of the tube 11 in a lengthwise direction of the needle 12. The slits 121 divide the body of the needle 12 into a plurality of needle strips 122. Each of the slits 121 has a given length with a top end and a base end opposite the top end. The top end of each of the slits 121 is in coincidence with the top end of the needle 12, while the base end of each of the slits 121 faces the base end of the tube 11. Each of the slits 121, as can be seen from FIGS. 1(a) to 1(c), is defined by an elongated opening which has a width gradually narrowing from the base end to the top end thereof. Specifically, each of the slits 121 has, as illustrated in FIG. 1(a), a base end width W₀ and a top end width W₁ which is smaller than the base end width W₀. The top end width W₁ is 0 mm so that top portions or pointed tips of the needle strips 122 are placed in contact with each other. The needle 12 also has a top opening whose diameter D1 is smaller than a width W₂ of a piece of physiological tissue, that is, a hair graft 100, as illustrated in FIGS. 5(a) to 5(g), containing a hair root which is to be extracted or removed by the hair implanter 10. The width W₂ of the hair graft 100 may be set to a statistical standard width of typical hair grafts.

In operation, the shaft 13 is first pushed from an initial position, as illustrated in FIG. 1(a), to the top of the tube 11. The shaft 13 travels within the tube 11 and then contacts, as illustrated in FIG. 1(b), the inner surfaces of the needle strips 122. The shaft 13 is further pushed in sliding contact with the inner surfaces of the needle strips 122. This will cause, as illustrated in FIG. 1(c), the needle strips 122 to be flexed radially outwardly to open the tip of the needle 12 in a leverage mode where the base ends of the slits 121 are pivot points (i.e., fulcrums) P1, and points of contact between the shaft 13 and the needle strips 122 are effort points P2. Each of the slits 121 is so shaped that an inside end (i.e., a right end, as viewed in FIGS. 1(a) to 1(c)) of the slit 121 is located more deeply or closer to the base end of the tube 11 (i.e., the base end of the body of the hair implanter 10) than a point where the tip of the shaft 13 first contacts the inner surface of the slit 121, that is, a contact point P2 of the tip of the shaft 13, as illustrated in FIG. 1(b), is.

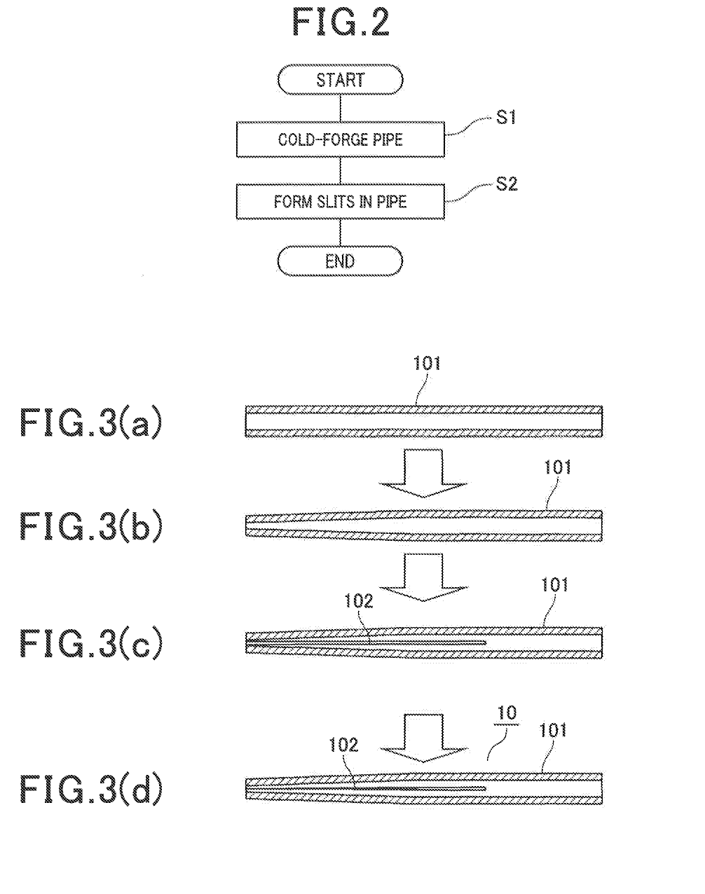

A production method of the hair implanter 10 will be described below with reference to FIGS. 2 to 3(d).

The hair implanter 10 is made by means of compression molding such as cold forging in which metal is compressed at temperatures (e.g., room temperatures) below a recrystallization temperature thereof.

Specifically, in step S1, a hollow cylindrical pipe 101, as illustrated in FIG. 3(a), is prepared. The hollow cylindrical pipe 101 is made of, for example, stainless steel. Next, a nose portion of the hollow cylindrical pipe 101 which is to be the needle 12 of the hair implanter 10 is, as illustrated in FIG. 3(b), cold-forged into a tapered form.

Subsequently, in step 52, two or more slits 102 are formed, as illustrated in FIG. 3(c), in a portion of the hollow cylindrical pipe 101 which ranges from the tip to a non-cold forged portion of the hollow cylindrical pipe 101 which is to be the tube 11 of the hair implanter 10. The slits 102 may be cut using the so-called wire electric discharging machine. Upon the formation of the slits 102, the residual stress which has been created by the cold-forging in the nose portion of the hollow cylindrical pipe 101 will cause, as illustrated in FIG. 3(d), strips of the hollow cylindrical pipe 101 into which the nose portion is divided by the slits 102, that is, which are to be the needle strips 122 of the hair implanter 10 to shrink inwardly until tip portions of the slits 102 are closed. This completes the hair implanter 10.

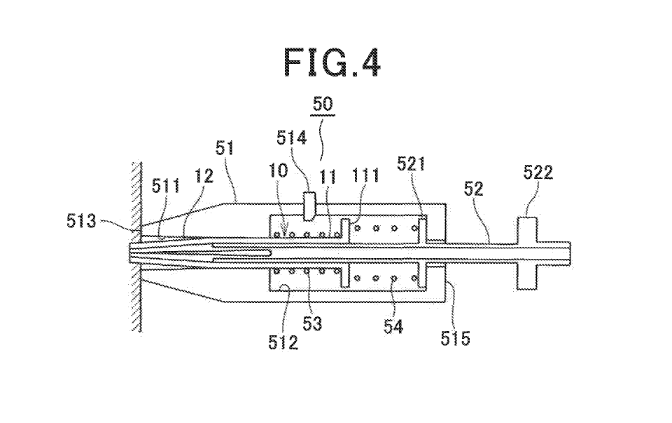

FIG. 4 demonstrates an example of the hair implanter 10 in use. The hair implanter 10 is installed inside a hair transplanter 50. Specifically, the hair transplanter 50 includes a casing or outer shell 51, a plunger or shaft 52, a coil spring 53, and a return spring 54. The shaft 52 works as the shaft 13, as described above. The hair implanter 10 is disposed within the shell 51 to be slidable in a lengthwise direction thereof. The hair implanter 10 has a flange 111 expanding outwardly from, the base end of the tube 11.

The shell 51 has formed therein a nose hole 511 extending straight in an axial direction thereof. The nose hole 511 opens at the tip end of the shell 51. The shell 51 also has formed in a base end portion thereof a cylindrical inner chamber 512 which is greater in diameter than the nose hole 511 and communicates with the nose hole 511. The tube 11 and the needle 12 of the hair implanter 10 are disposed to be slidable within the nose hole 511. The flange 111 of the hair implanter 10 is mounted to be slidable within the inner chamber 512. The shell 51 has a tapered head with a tip surface 513. The tip surface 513 is even or flat and to be placed in touch with, for example, skin to be transplanted. The shell 51 has a stopper 514 extending radially through a side wall thereof. When the needle 12 of the hair implanter 10 is inserted into, for example, skin and reaches a maximum permissible depth, the flange 111 of the hair implanter 10 engages or snaps on the stopper 514 to hold the hair implanter 10 from moving back to the base end of the hair transplanter 50.

The shaft 52 is made of a hollow cylindrical tube with a hole extending through the length thereof. The shaft 52 has a first flange 521 located near the center of the outer surface thereof and a second flange 522 disposed closer to the base end thereof than the first flange 521 is. The first flange 521 comes in contact with the inner surface of a base end wall (i.e., a bottom wall) 515 of the shell 51 when the hair implanter 10 is drawn to the initial. position (i.e., the rightmost position in the drawing) within the shell 51 of the hair transplanter 50. The second flange 522 comes in contact with the outer surface of the base end wall 515 when the shaft 52 moves to the top end of the hair implanter 10 to push the hair implanter 10, and the hair implanter 10 reaches the maximum permissible depth. The maximum permissible depth may be selected as a function of the size of the hair graft 100.

The spring 53 is disposed within the inner chamber 512 in tough with the outer peripheral surface of the tube 11 of the hair implanter 10 in engagement with the top end surface of the inner chamber 512 and the front surface of the flange 111. The spring 53 works to urge the hair implanter 10 to the base end of the shell 51.

The return spring 54 is wound around the shaft 52 within the inner chamber 512 in engagement with the flange 111 of the hair implanter 10 and the first flange 521 of the shaft 52. The return spring 54 works to produce spring pressure which urges the shaft 52 toward the base end of the shell 51. When thrusting pressure acting on the shaft 52 is released, it will cause the shaft 52 to be urged by the return spring 54 toward the base end (i.e., the right side in the drawing) of the shell 51, so that the shaft 52 is moved back to the initial position thereof. The return spring 54 is so selected as to the pressure which is lower in compressed strength than the spring 53.

In operation, when the shaft 52 of the hair transplanter 50 is pushed and advanced from the initial position, the return spring 52 is compressed. The hair implanter 10 is, thus, advanced by the spring pressure exerted by the return spring 52 and the thrusting pressure from by the shaft 52. The return spring 54 is, as described above, designed to be compressed by pressure which is weaker than that by which the spring 53 is compressed. The amount of advancement of the shaft 52 is, thus, greater than that of the hair implanter 10, thereby causing the top of the shaft 52 to contact the inner surface of the needle strips 122, so that the needle strips 122 expand radially outwardly and open. Simultaneously, the hair implanter 10 is inserted into a harvesting area of the skin. When the amount by which the hair implanter 10 has been inserted into the skin reaches the maximum permissible depth, the flange 111 of the hair implanter 10 snaps into the stopper 514. The stopper 514 holds the implanter 10 from being returned back toward the initial position thereof. Subsequently, the shaft 52 stops being thrust. This causes only the shaft 52 to be returned by the return spring 54 back to the initial position thereof.

In order to open the needle strips 122 elastically along a desired path following the advance of the shaft 52, the spring constant K1 of the needle strips 122, the spring constant K2 of the spring 53, and the spring constant K3 of the return spring 54 are so selected as to meet relations below.

K2+(K1+K3)/R=0 (1)

R=X/Xc (2)

where X is the amount of movement of the hair implanter 10, Xc is the amount of movement of the shaft 52 relative to the hair implanter 10, and R is a ratio of the amount of movement of the hair implanter 10 to that of the shaft 52.

How to extract or harvest the hair graft 100 with a root of hair using the hair transplanter 50 and implant the hair graft 100 in the recipient site will be described below with reference to FIGS. 5(a) to 5(g). FIGS. 5(a) to 5(d) represent a harvesting operation. FIGS. 5(e) to 5(g) represent a transplanting step operation.

First, a pre-insertion step, as illustrated in FIG. 5(a), is performed. Specifically, the tip surface 513 of the shell 51 of the hair transplanter 50 is placed in contact with the skin of, for example, scalp of a recipient of hair transplantation. The hair transplanter 50 is positioned so as to surround an area of the skin containing a hair to be harvested by the needle strips 122 of the hair implanter 10.

Next, an insertion step, as illustrated in FIG. 5(b), is performed. Specifically, the shaft 52 is pushed in a direction, as indicated by an arrow A, to insert the needle 12 into the harvesting area of the scalp skin. The needle 12 advances in the scalp skin. The needle strips 122 are gradually expanded from initial positions thereof following the advance of the shaft 52. When the needle 12 of the hair implanter 10 reaches the maximum permissible depth, the needle strips 122 will expand most greatly (i.e., to an upper limit). Simultaneously, the stopper 514 snap-fit engages the flange 111 of the hair implanter 10 to hold the hair implanter 10 from moving back to the initial position thereof.

A holding step, as illustrated in FIG. 5(c), is performed. Specifically, the thrusting pressure acting on the shaft 52 is released. This causes the shaft 52 to be urged by the return spring 54 in a direction, as indicated by an arrow B (i.e., toward the base end of the shell 51). The hair implanter 10 is, as described above, held from being returned back to the initial position thereof and thus kept in the inserted position. Only the shaft 52 is moved to the base end of the hair transplanter 50. The air may he sucked from inside the shaft 52 from the base end thereof to keep the inner chamber of the hair implanter 10, especially the inner chamber of the needle 12 at the negative pressure, thereby facilitating the ease with which the contraction of the needle strips 122 is accelerated to cut the hair graft 100 out of the scalp tissues and ensuring the stability in grasping the hair graft 100 inside the top end of the needle 12.

An extracting step, as illustrated in FIG. 5(d), is performed. Specifically, the whole of the hair transplanter 50 is drawn to pull the needle 12 out of the scalp, thereby completing the disconnection of the hair graft 100 from the scalp tissues with the hair graft 100 put in the needle 12 of the hair implanter 10.

Upon completion of the above harvesting operation, as described above, to obtain the hair graft 100, the transplanting operation is executed.

First, an insertion/implanting step, as illustrated in FIG. 5(e), is performed. Specifically, the hair transplanter 50 in which the hair graft 100 is retained is pushed to insert the needle 12 into the scalp skin, thereby implanting the hair graft 100 into the recipient site. The stopper 514 of the hair transplanter 50 is kept in engagement with the flange 111 of the hair implanter 10. In other words, the needle 12 protrudes farther away from the tip of the shell 51 of the hair transplanter 50. The insertion of the needle 12 into the scalp skin is, therefore, achieved by thrusting the whole of the hair transplanter 50 until the tip surface 513 touches the scalp skin. The transplanting operation may alternatively be initiated with the stopper 514 being released from the flange 111 of the hair implanter 10. Specifically, like in the harvesting operation to obtain the hair graft 100, the whole of the hair transplanter 50 is thrust forward while pushing the shaft 52 to insert the needle 12 into the scalp.

Next, a graft-releasing step, as illustrated in FIG. 5(f), is performed. Specifically, the shaft 52 is thrust until it contacts the base end of the outer shell 51 of the hair transplanter 50 to maximize the expansion of the needle strips 122. Fresh air may be supplied into the shaft 52 from the base end thereof to keep the inner chamber of the needle 12 at the positive pressure, thereby facilitating the ease with which the hair graft 100 is released from the head of the needle 12.

Finally, a drawing step, as illustrated in FIG. 5(g), is performed. Specifically, the whole of the hair transplanter 50 is drawn from the scalp with the needle strips 122 kept expanding, thereby completing the transplantation of the hair graft 100 into the recipient site of the scalp.

As described above, the hair implanter 10 has the slits 121 to define the needle strips 122. The slits 121 extend toward the base end of the tube 11 across the point P2 at which the shaft 52 (or the shaft 13) advances inside the tube 11 and first contacts the inner surface of the needle strips 122, thereby causing the needle strips 122 to bend or expand radially outwardly to open the tip of the needle 12 in the leverage mode where the base end of each of the slits 121 is the pivot point (i.e., fulcrum) P1, and the point of contact between the shaft 52 and each of the needle strips 122 is the effort point P2. This enables the needle strips 122 to be expanded to open the tip of the needle 12 smoothly by pushing the shaft 52 (or the shaft 13) forward with a small degree of force.

Each of the slits 121 is so shaped as to have a width which gradually decreases from the base end of the needle 12 and reaches zero at the tip of the needle 12, so that the tips of the needle strips 122 are placed in contact with each other. The needle strips 122 are so formed as to elastically contract in the radial direction of the needle 12 further when the tips of them are placed in contact with each other, thereby enabling the tip of the needle 12 to be pushed against the surface of the scalp to make the incision in the scalp skin easily and also ensuring the stability in holding the hair graft 100 extracted from the scalp skin.

The hair transplanter 50 is, as described above, equipped with the return spring 54, thus enabling the shaft 52 to be moved back to the initial position thereof only by releasing the pressure acting on the shaft 52 after the completion of the insertion step of FIG. 5(b). This permits an operator to handle the hair transplanter 50 in one of his or her hands, thus facilitating the ease of sampling the hair graft 100.

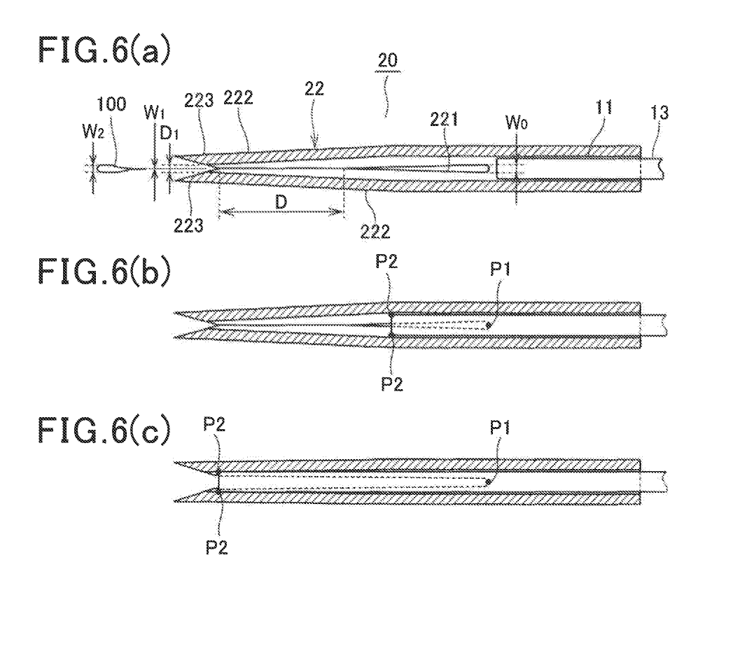

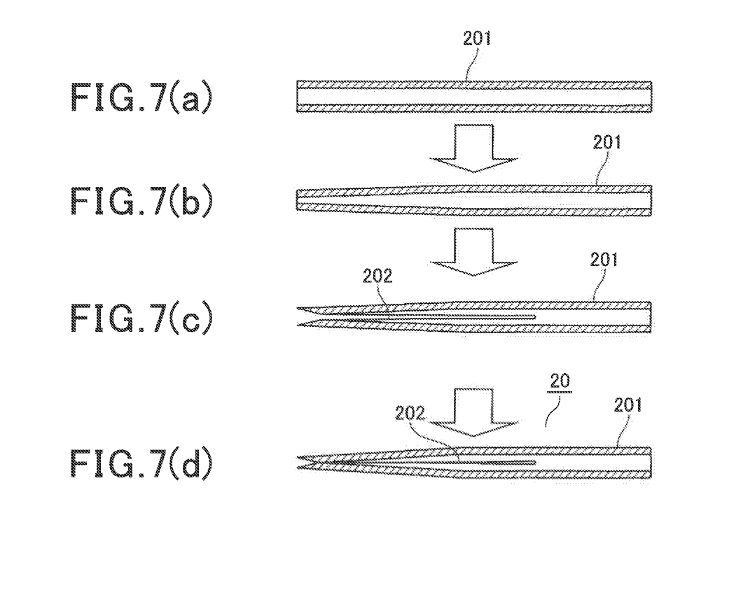

FIGS. 6(a) to 7(d) illustrate hair implanter 20 of the second embodiment. The same reference numbers as employed in the first embodiment will refer to the same parts, and explanation thereof in detail will be omitted here.

The hair implanter 20, like in the first embodiment, includes a needle 22. The needle 22 has slits 221 formed therein. Each of the slits 221 extends straight from the tip toward the base end of the needle 22 along the lengthwise direction of the needle 22. The slits 121 divide the body of the needle 12 into a plurality of needle strips 222. Each of the needle strips 222, as clearly illustrated in FIGS. 6(a) to 6(c), has a wedge-shaped blade edge 223 which is of a tapered shape or wedge shape. Specifically, each of the slits 221 has, as illustrated in FIG. 6(a), a base end width W₀ and a top end width W₁ which is smaller than the base end width W₀. In this embodiment, the top end width W₁ is 0 mm so that middle portions of the needle strips 222 each of which excludes the blade edge 223, that is, extends from the base end of the blade edge 223 in the axial direction of the needle 22 by a given distance D may be placed in contact with each other. The needle 12 also has a top opening whose diameter D₁ is smaller than the width W₂ of the hair graft 100 to be extracted or harvested by the hair implanter 20. The top opening is a graft inlet defined by, for example, the base ends of the blade edges 223 in a direction perpendicular to the length of the needle 22. In other words, the top opening is a minimum opening defined by the blade edges 223 of the needle strips 222.

In operation, the shaft 13 is first pushed from the initial position, as illustrated in FIG. 6(a), to the top of the tube 11. The shaft 13 travels within the tube 11 and then contacs, as illustrated in FIG. 6(b), the inner surfaces of the needle strips 222. The shaft 13 is further pushed in sliding contact with the inner surfaces of the needle strips 222. This will cause, as illustrated in FIG. 6(c), the needle strips 122 to be flexed radially outwardly to open the tip of the needle 12 in a leverage mode where the base ends of the slits 221 are pivot points (i.e., fulcrums) P1, and points of contact between the shaft 13 and the needle strips 222 are effort points P2. Each of the slits 221 is so shaped that an inside end (i.e., a right side end, as viewed in FIGS. 6(a) to 6(c)) of the slit 221 is located deep or closer to the end of the tube 11 than a point where the tip of the shaft 13 first contacts the inner surface of the slit 121, that is, a contact point P2 of the tip of the shaft 13, as illustrated in FIG. 6(b), is.

A production method of the hair implanter 20 will be described below with reference to FIGS. 7(a) to 7(d).

The hair implanter 20 is made by cold forging. Specifically, a hollow cylindrical pipe 201, as illustrated in FIG. 7(a), is prepared. The hollow cylindrical pipe 201 is made of, for example, stainless steel. Next, a nose portion of the hollow cylindrical pipe 201 which is to be the needle 22 of the hair implanter 20 is, as illustrated in FIG. 7(b), cold-forged into a tapered form.

Subsequently, two or more slits 202 are formed, as illustrated in FIG. 7(c), in a portion of the hollow cylindrical pipe 201 which ranges from the tip to a non-cold forged portion of the hollow cylindrical pipe 201 which is to be the tube 11 of the hair implanter 20. The slits 202 may be cut using a so-called wire electric discharging machine.

The structure of each of the slits 202 will be described below. The slits 102 of the hair implanter 10 of the first embodiment extend straight in the axial direction of the cylindrical pipe 101, however, the slits 202 of the hair implanter 20 are designed in a Y-shape. Specifically, each of the slits 202 is made up of three sections which are different in width from each other, in other words, inclination of side edges thereof relative to the axis (i.e., the length) of the slit 202. The three sections include a first section (i.e., a top portion), a second section (i.e., a middle portion), and a third section (i.e., a base portion). The first section is defined by the blade edges 223 and spreads radially at the greatest angle, in other words, has side edges (i.e., the blade edges 223) which extend at the greatest inclination to the axis of the slit 202. The second section is the middle portion. of the slit 202 except the top portion (i.e., the first section) and the base portion (i.e., the third section) of the slit 202 and where the needle strips 222 are in contact abutment with each other, in other words, has side edges which extends at a smaller inclination to the axis of the slit 202 The third section has side edges extending substantially parallel to the axis of the slit 202. Each of the slits 202 may alternatively be formed in a V-shape, in other words, made up of two sections which are different in inclination of side edges to the axis of the slit 202 or include either two of the first, second, and third sections, as described above.

Upon the formation of the slits 202, the residual stress which has been created by the cold-forging in the nose portion of the hollow cylindrical pipe 201 will cause, as illustrated in FIG. 7(d), strips of the hollow cylindrical pipe 201 into which the nose portion is divided by the slits 202, that is, which are to be the needle strips 222 of the hair implanter 20 to elastically shrink inwardly until tip portions of the slits 202 are closed completely. This finishes the hair implanter 20.

As described above, the hair implanter 20 has the slits 221 to define the needle strips 222. The slits 221 extend up to the tip of the needle 22 across the point P2 at which the shaft 13 advances inside the tube 11 and first hits the inner surface of the needle strips 222, thereby causing the needle strips 222 to expand radially outwardly to open the tip of the needle 22 in the leverage mode where the base end of each of the slits 221 is the pivot point (i.e., fulcrum) P1, and the point of contact between the shaft 13 and each of the needle strips 222 is the effort point P2. This enables the needle strips 222 to be expanded to open the tip of the needle 22 smoothly by thrusting the shaft 13 forward with a small degree of force.

The structure of the slits 221 permits an air gap between adjacent two of the needle strips 222 to be minimized, thereby ensuring the stability in keeping the inner chamber of the needle 22 at negative pressures. The needle 22 has the blade edges 223, thereby minimizing the resistance against the insertion of the needle 22 into the skin, which facilitates the ease of harvesting the hair graft 100 from the scalp or transplanting it to the recipient site.

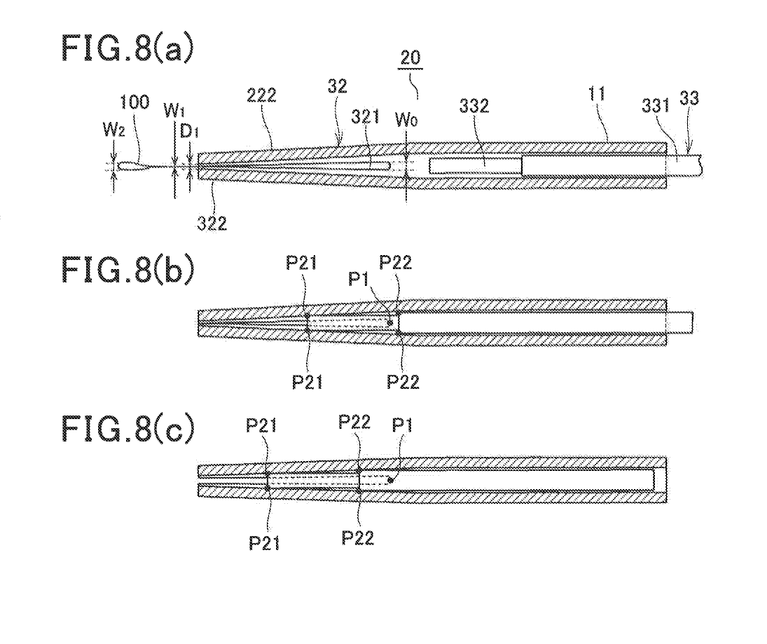

FIGS. 8(a) to 8(c) illustrate hair implanter 30 of the third embodiment. The same reference numbers as employed in the first embodiment will refer to the same parts, and explanation thereof in detail will be omitted here.

The hair implanter 30, like in the first embodiment, includes a needle 32 and a shaft 33. The shaft 33, like in the first embodiment, works as a plunger and includes an extension or nose 332 which extends from a major body 331 of the shaft 33 and is smaller in diameter than the major body 331. The needle 32 has slits 321 formed therein. Each of the slits 321 extends straight from the tip toward the base end of the needle 32 along the lengthwise direction of the needle 32. The slits 321 divide the body of the needle 32 into a plurality of needle strips 322. The slits 321, as clearly illustrated in FIGS. 7(a) to 7(c), are similar in shape to the slits 121 of the hair implanter 10 in the first embodiment, however, different in length therefrom. Specifically, each of the slits 321 extends from the tip toward the base end (i.e., the right side, as viewed in the drawing) of the needle 32 across the point P21, as can be seen in FIG. 8(b), at which the nose 332 of the shaft 33 advances inside the tube 11 and first hits the inner surface of the needle strips 322, however, terminates before the point P22 at which the corner of the major body 331 of the shaft 33 first hits the inner surface of the needle strips 322. In other words, each of the slits 321 extends from the tip of the needle 32 and ends between the points P21 and P22, as illustrated in FIG. 8(b).

The slits 321 are shorter than the slits 121 or 122 in the first or second embodiment, thereby resulting in an increase in spring constant of the needle strips 322. This requires a greater degree of pressure than that in the first and second embodiment to elastically open the needle strips 322. In order to alleviate such a drawback, the shaft 33 is designed to include the nose 332 working as a sub-plunger which is smaller in diameter than the major body 331.

In operation, the shaft 33 is first pushed from the initial position, as illustrated in FIG. 8(a), to the top of the tube 11. The shaft 33 travels within the tube 11 and then hits, as illustrated in FIG. 8(b), the needle strips 322. More specifically, the corner of the nose 332 of the shaft 33 hits the inner surfaces of the needle strips 322. The shaft 33 continues to be pushed in sliding contact with the inner surfaces of the needle strips 322. The corner of the major body 331 (i.e., the shoulder of the shaft 33) then contacts the inner surfaces of the needle strips 322. Further movement of the shaft 33 will cause, as illustrated in FIG. 8(c), the needle strips 322 to be expanded radially outwardly to open the tip of the needle 32 in a leverage mode where the base ends of the slits 321 are pivot points (i.e., fulcrums) P1, and points P21 of contact between the nose 332 of the shaft 33 and the inner surfaces of the needle strips 322 and points P22 of contact between the major body 331 and the inner surfaces of the needle strips 322 are effort points. Specifically, the needle strips 322 are opened outwardly by pushing force at the two effort points.

As apparent from the above discussion, the slits 321 are so designed as to have a length extending in the lengthwise direction of the hair implanter 30 which is smaller than that in the first and second embodiments, thereby resulting in an increase in pressure for cutting a tissue from the scalp or holding it. The spring constant of each of the needle strips 322 is increased as compared with that in the first or second embodiment, however, the shaft 33 is designed to have the two effort points P21 and P22 where the pressures are produced to expand the needle strips 322, thus permitting the tip of the needle 32 to be opened smoothly by pushing the shaft 33 forward with a small degree of force.

While the present invention has been disclosed in terms of the preferred embodiments in order to facilitate better understanding thereof, it should be appreciated that the invention can be embodied in various ways without departing from the principle of the invention. Therefore, the invention should be understood to include all possible embodiments and modifications to the shown embodiments which can be embodied without departing from the principle of the invention as set forth in the appended claims.

What is claimed is:

1. A hair implanter comprising:
a hollow cylindrical tube in which a shaft is to be moved in an axial direction of the tube, the tube having a top end and a base end opposite the top end;
a hollow cylindrical needle having a tip and a base end opposite the tip, the needle extending at the base end thereof from the tip end of the tube, the needle tapering toward the tip; and
a plurality of slits extending from the tip of the needle in an axial direction of the needle to divide a body of the needle into a plurality of needle strips, the needle strips being so shaped that when the shaft is moved toward the top of the tube, the shaft contacts inner surfaces of the needle strips, and when the shaft is further advanced in sliding contact with the inner surfaces of the needle strips, the needle strips will elastically bend radially outwardly, each of the slits extending toward the base end of the tube across a point at which the shaft hits the inner surfaces of the needle strips.

2. A hair implanter as set forth in claim 1, wherein each of the slits has a given length with a top end and a base end and also has a width which gradually narrows from the base end to the top end of the slits.

3. A hair implanter as set forth in claim 1, wherein the slits are so shaped that portions of the needle strips which are closer to the top end thereof than to the base end thereof are placed in touch with each other.

4. A hair implanter as set forth in claim 3, wherein the portions of the needle strips placed in touch with each other are middle portions of the needle strips excluding the top end of the needle.

5. A hair implanter as set forth in claim 3, wherein the portions of the needle strips each has a given length extending in the axial direction of the needle.
